(19) United States
(12) Patent Application Publication
(10) Pub. No.: US 2016/0331437 A1
(43) Pub. Date: Nov. 17, 2016

(54) PLASMA DEVICE

(71) Applicants: Thomas Bickford Holbeche, Church Crookham (GB); Rodney Stewart Mason, Blakpill (GB)

(72) Inventors: Thomas Bickford Holbeche, Church Crookham (GB); Rodney Stewart Mason, Blakpill (GB)

(21) Appl. No.: 15/112,693

(22) PCT Filed: Jan. 22, 2015
(86) PCT No.: PCT/GB2015/000015

§ 371 Date: Jul. 20, 2016

(30) Foreign Application Priority Data
Jan. 23, 2014 (GB) 1401151.4

(51) Int. Cl. A61B 18/04; H05H 1/24; H05H 1/52; A61B 18/12; A61N 1/44

(52) U.S. Cl. CPC A61B 18/042; A61B 18/1206; A61N 1/44; H05H 1/52; H05H 1/2406; H05H 2245/122; H05H 2245/125; H05H 2001/2412; H05H 2001/2456

(57) ABSTRACT

A plasma-generation device for applying plasma to a human body, having a reservoir containing a gas; a plasma zone in fluid connection with the reservoir, and means for generating a plasma by electrical discharge in the plasma zone. A means for generating free electrons is provided downstream of the reservoir and upstream of the plasma zone.

present disclosure relates to a non-thermal plasma treatment device. In particular, the disclosure relates to a reduced energy consumption device for the production of a so-called “cold plasma”.

A gas is normally an electric insulator. However, when sufficient thermal energy is supplied to a gas or, alternatively, a sufficiently large potential difference is applied across a gap containing a gas, then it will breakdown and conduct electricity. This is because the electrically neutral atoms or molecules of the gas have been ionised to form electrons and positively charged ions. This ionised gas is a plasma.

When the ionisation is driven by a large potential difference, the momentum transfer between the light electrons and the heavier gas molecules and plasma ions is not very efficient. Therefore, the bulk of the energy that is supplied to form the plasma is supplied to the electrons. As a result, ionised gases, particularly at low gas pressures and charged particle densities, are described as “cold” or non-thermal. This means that the constituents e.g. the electrons, ions and gas molecules are each in thermal equilibrium only with similar mass species.

Such non-thermal plasmas are well known for use in destroying bacteria. For this reason, it is known to use non-thermal plasma in various forms of dental surgery. Due to the restrictions when operating in a patient's mouth, such plasma devices typically rely on a flow of gas between two electrodes to produce the plasma which can be directed onto the treatment area. The non-thermal production of the plasma provides a plasma gas having a temperature which is tolerable for the patient. WO2013040476 discloses a device for generating plasma which uses a flow of Helium.

Recently a number of proposals have been put forward to provide a system for the generation of non-thermal (also known as non-equilibrium) gas plasma in the industrial, dental, medical, cosmetic and veterinary fields. Non-thermal gas plasma generation can be employed to promote coagulation of blood, cleaning, sterilisation, removal of contaminants from a surface, disinfection, re-connection of tissue and treatment of tissue disorders without causing significant thermal tissue damage. The plasma itself may be applied to a surface to be treated or may act as a precursor of a reactor for a modified gaseous specifies that is applied to the surface.

One of the key requirements for the uptake of such a device is that it has relatively low power consumption so that it can be made of size that can be readily used in a domestic or in-surgery environment.

As a result of this, the system should be as efficient as possible. The present invention is directed to a design of the plasma device which allows a lower power electric discharge for plasma generation.

It is an object of the present invention to provide an improved approach to the generation of plasma, tackle the drawbacks associated with the prior art, or at least provide a commercially viable alternative thereto.

According to a first aspect, there is provided a plasma-generation device for applying plasma to a human body, the device comprising:

a reservoir containing a gas;

a plasma zone in fluid connection with the reservoir;

means for generating a plasma by electrical discharge in the plasma zone and

wherein downstream of the reservoir and upstream of the plasma zone there is provided a means for generating free electrons.

The present disclosure will now be described further. In the following passages different aspects/embodiments of the disclosure are defined in more detail. Each aspect/embodiment so defined may be combined with any other aspect/embodiment or aspects/embodiments unless clearly indicated to the contrary. In particular, any feature indicated as being preferred or advantageous may be combined with any other feature or features indicated as being preferred or advantageous.

The present invention relates to a plasma-generation device. That is, the device is designed to produce a plasma from the ionisation of a gas. The device is especially for producing a non-thermal plasma, as discussed herein. The plasma produced preferably has a temperature of less than 50° C., more preferably less than 48° C., more preferably less than 45° C., and most preferably from 37 to 42° C. It will be appreciated that for certain treatments, especially for hair treatment, temperature may suitably be at even higher temperatures. The device is suitable for applying plasma to a human body, which applies a number of constraints since thermal plasma production devices are clearly unsuitable. Furthermore, the production levels of UV, electrical stimulation and active species must be at levels which do not cause undue harm to a patient. It is noted that the human pain threshold for temperature is typically around 48° C.

The device described herein is preferably hand-held. By hand-held, it is meant that at least the treatment application head is sized and configured such that it can be readily manipulated and controlled with one hand. Examples of hand-held devices include hair-brushes, hair-driers, foot-spa, hair-tongs, toothbrushes and the like. The treatment application head may be tethered to a power supply and/or a gas reservoir. Alternatively the treatment head may be fixed or pivotable with relation to an area to be treated. The device may also, for example, take a form such as a foot spa to allow ready treatment of an infected foot.

The ideal form for home use by a consumer is an entirely self-contained hand held device. This would have an internal battery as a power source and rely upon interchangeable gas canisters which can be clipped into the device. Nonetheless, for reasons of power requirements, it may be easier to have a mains power lead attached to the device.

Especially when the device is to be used by a professional, such as in a hair or nail salon, or by a doctor, podiatrist, or the like, it may be easier to have the hand-held device tethered to a power supply and a larger gas tank. This makes it easier for the professional to use since they do not need to change the gas tank/cartridge/canister often.

Preferably the power supply comprises a battery integrated into the hand-held device. That is, preferably the plasma-generation device is entirely independent and does not require a tether to a power supply. This increases the utility of the device in-so-far as it can be more accurately applied and can be used in a wider range of environments, such as bathrooms.

The use of a hand-held device as discussed herein has a large number of advantages. The provision of the plasma keeps the device sterile and it can be readily reused for multiple patients. In addition, the plasma produces a ready supply of active gas species which provide the treatments discussed herein. The active gas species are further supplemented by the temperature, UV light and electrical stimulation which are associated with the plasma production process.

The plasma treatment device comprises a reservoir containing gas. The reservoir acts as a source of gas from which a plasma is generated. The reservoir contains a source of pressurised gas which can be supplied to the plasma zone as the treatment application portion of the device. The gas will typically be stored in a tank (up to approximately 200 L) for professional use, or in replaceable and/or rechargeable canisters of cartridges for home use. The use, design and requirements for such sources of gas are well known in the art.

The gas is preferably Helium, Argon, Neon, Krypton, or Hydrogen, or mixtures of two or more thereof.

The reservoir is in fluid communication with a plasma zone within which plasma is created for treatment. In some embodiments the plasma zone is within the device and a flow of the plasma which is created leaves the device to provide the treatment. In other embodiments the plasma is formed directly at the site to be treated. The plasma zone includes means for generating a plasma by electrical discharge therein.

The device comprises a means for generating a plasma by electrical discharge through the gas. This can be achieved by one of several different approaches.

According to a first approach, the means for generating a plasma comprises comprises a power supply and a dielectric electrode for placing in proximity to a human body, and wherein, in use, the plasma zone is formed between the dielectric electrode and a surface of a human body. The provision of a high voltage drop between the dielectric electrode and the human body leads to the production of a plasma between the dielectric electrode and the body. This is an effective way to treat a large area. The device of the present invention would preferably be configured such that the gases discussed herein can be flowed into the space formed between the dielectric electrode and the body, preferably at a relatively low flow rate, across substantially the whole area of the electrode.

According to a second approach the means for generating a plasma comprises a power supply, and first and second electrodes, and wherein, in use, the plasma zone is formed between the first and second electrodes and wherein a flow of gas from the reservoir through the plasma zone provides a flow of plasma to contact a surface of a human body. The provision of a high voltage drop between the two electrodes will cause the production of a plasma by ionising the gas provided. In this embodiment the gas flow will typically be greater so that the plasma flows out from between the electrodes and can be applied to a treatment area.

According to a third approach, the means for generating a plasma is a so-called surface micro discharge device. This comprises a power supply and first and second electrodes sandwiching a dielectric material. In use, a plasma zone is formed adjacent a surface electrode which can be held close to a surface of a human body. The provision of a high voltage drop between the electrodes leads to the production of a plasma across the area and, indeed, the electrode close to the treatment area will typically be a wire mesh. This is an effective way to treat a large area. The device of the present invention would preferably be configured such that the gases discussed herein can be flowed into the space formed between an external electrode on the device and the body, preferably at a relatively low flow rate, across substantially the whole area of the electrode.

Preferably the means for generating a plasma operates at a voltage of from 2-15 kV, preferably from 3 to 10 kV and most preferably about 5 kV. These levels of voltage can be achieved in a hand-held device and still produce a suitable level of plasma generation. The power range of the device is preferably 1-100 Watts AC at a high frequency of 10-60 KHz. Alternatively, power may be delivered as high frequency pulsed DC fast rise time square waveforms.

Preferably the gas is supplied through the means for generating a plasma at a flow rate of less than 5 l/min, preferably less than 2.5 l/min, more preferably less than 1.5 l/min, preferably from 0.1 to 1 l/min, preferably from 0.01 to 0.5 l/min. The gas flow rate for area treatments as discussed above will typically be lower than required for point treatments which require the production of a targeted jet of plasma. The flow rates for treatments which produce a plasma between a dielectric electrode a treatment are of a patient are preferably from 0.01 to 0.1 l/min. The flow rates for treatments which produce a plasma between two electrodes and rely on the gas flow to carry the plasma to a treatment are preferably from 0.5 to 2.5 l/min.

Preferably the device takes the form of a hair straightener, a toothbrush, a foot-spa or a hair-brush. In these recognisable forms, the consumer is already familiar with the usage requirements and application techniques required to employ the device. This avoids any hurdle to application. More particularly, these application devices are suitable for the application of the plasma to the regions that specifically require treatment, such as the hair or teeth of a user.

The present inventors have found that it is possible to decrease the powder required to provide for the continuous production of plasma by use of an electron seeding technique. This technique relies on the generation of free electrons by use a device distinct from the means for generating a plasma by electrical discharge. This secondary device provides the free electrons which reduces the threshold energy required for plasma generation. This technique can be used in combination with the designs of plasma treatment unit discussed herein.

Specifically, the inventors have found that in an AC discharge, the discharge strikes as the increasing voltage in the first quarter cycle reaches a threshold (spark) value. This leads to an avalanche ionisation as it becomes exponentially easier for further ionisation to occur. Thus the gas becomes partially ionised and the voltage required to sustain the discharge drops, and if conditions are right the ions and electrons created will form a temporary glow discharge (GD) plasma.

However, as soon as the voltage in the second quarter drops below the critical level, ionisation will cease and the plasma will rapidly decay.

As the voltage starts to rise again in the third quarter, the ionisation begins again at the threshold. The inventors found that if there are electrons still around from the previous quarter, the threshold will be lower. The discharge then falls off again in the fourth quarter and grows again in the first quarter of the next cycle as the cycles continue.

The plasma, or ion-electron population, therefore waxes and wanes at twice the frequency of the applied voltage, and the plasma is in fact a pulsed plasma. If the frequency is high enough, however, the plasma hardly has time to wane before the next ionisation pulse builds it up again, and the electrons already present (left over from the previous one) make that pulse easier to generate. Soon, the oscillating plasma population builds up to a steady state average level, determined by the gas, the frequency and the applied voltage.

The inventors found that the steady state population very much depends on the time constant for the plasma when it is decaying. For a given gas and geometry, the decay rate is determined by the sum of two effects: the rate of recombination of electrons and ions, and the rate of their diffusion to the walls of the discharge tube. These both depend on the pressure; but the former is directly dependent and the latter is inversely dependent, and so the effect on one may help cancel out the effect on the other. For a given decay rate, the higher the frequency, then the more continuous is the plasma population. In general, at low pressure, it appears that the discharge is effectively continuous for frequencies≧100 kHz. That is why, in general, rf plasmas are used, rather than for instance 50 Hz ac. In rf discharges it appears that only the applied voltage makes any difference to the discharge power; it is more or less independent of frequency.

There are two problems with regard to minimising power consumption: (i) the voltage of the operating power supply is determined by highest field (volts per cm) required by the threshold ionisation value on the first pass, after which it can subsequently operate at a much lower voltage; (ii) the need to deliver a uniform field over the whole cathode surface—this is spoilt by non-uniformity of the surface, gases adsorbed on the surface unevenly, particles of dust which serve as discharge points, wear and tear on the surface creating favoured paths.

One way around (ii) may be to provide a network of sharp edges, where the field becomes deliberately concentrated, but spread over the whole discharge space by using for example a fine stainless steel mesh as the metal electrode; the idea being to deliberately provide sharp fields which over-ride sharp fields created accidentally, as described above, and therefore is more consistent.

A necessary feature of all cold atmospheric plasmas seems to be that they are run at high power to get efficient initial ionisation, but in short bursts to keep the overall average power consumption low enough, so that the output gas remains thermally cold, whilst still carrying a therapeutic dose of (mainly) radicals, but also ions electrons and excited states. So the feeding power supply has to run at a much higher power than the average current drawn actually requires. The inventors realised that it would be beneficial, therefore, if it were possible to somehow ease the strike voltage requirement of the first few cycles of the discharge, and then to run the discharge continuously at an appropriate low level.

The inventors have now realised that this higher power can be avoided through the provision of seed electrodes.

The actual breakdown voltage for He is ˜150 V. Similarly, for air it is ˜300V. In practice, however, it is necessary to go up to 2.5 kV (884 V rms), to generate a plasma in air. The inventors theorised that this is because to get breakdown (and hence a discharge) you need the presence of at least one free electron. In reality it is probably more. These are provided by random ionisation events such as from background radiation or cosmic particles, and so at the threshold you could be waiting a relatively long time. However, the higher the voltage, then the more likely, when a free electron does appear, that discharge strikes. A high frequency can aid things further because when the electron is accelerated it gets slowed by (non-ionising) collisions with the gas. The higher the frequency then the sharper is the field during the ionisation quarter of the cycle. It therefore accelerates the electron in a shorter period of time, with less probability of collision before it reaches the ionisation energy, when it does eventually collide.

To avoid this problem the inventors sought for sources of free electrons which would lower the voltage required. By free electrons it is meant that the electrons are not bound to any atom or molecule.

In one embodiment the means for generating free electrons is a radiation source. Preferably the radiation source is or comprises the americanium. This is used in ionisation smoke detectors.

In one embodiment the means for generating free electrons is a UV lamp. UV lamps are well known in the art and include small UV LED lamps. The UV lamp serves to provide ionising photons.

In one embodiment the means for generating free electrons is an electrical filament. By passing a current through the filament there are thermionic emissions from the heated wire. Suitable low work-function wires are known. Advantageously, in the presence of the plasma gases described herein, and free of oxygen, the filament would have a long working life-time.

In one embodiment the means for generating free electrons comprises a pair of electrodes configured to provide a spark when a voltage is applied between them. The electrodes would preferably comprise a pair of spikes arranged in a duct between the reservoir and the plasma zone. A low current (preferably less than 1 μA) corona discharge or spark would require less than 10⁻³ watts power to provide a continuous low level of free electrons. This would be particularly advantageous for a hand-held low-power device. The spark or corona being at such a low current would pose no danger, or discomfort, to the user (through the gas), even though there is exposure to high voltage metal electrodes, but would provide the necessary seed electrons for ignition.

In use, the gas flows downstream from the reservoir to the means for generating free electrons. The free electrons are entrained in the gas flow to the plasma zone, whereby a plasma is generated by electrical discharge. The presence of the free electrons has been found to decrease the threshold energy required to initiate the plasma formation.

The device discussed herein is suitable for use in the treatment of nails, skin and hair. Suitable treatments include hair bleaching, nail fungus treatment, tooth whitening and the like.

### FIGURES

The present disclosure will be described in relation to the following non-limiting FIGURES, in which:

FIG. 1 shows a cross-sectional view of a plasma-generation device nozzle.

### EXAMPLES

The present disclosure will now be described in relation to the following non-limiting examples.

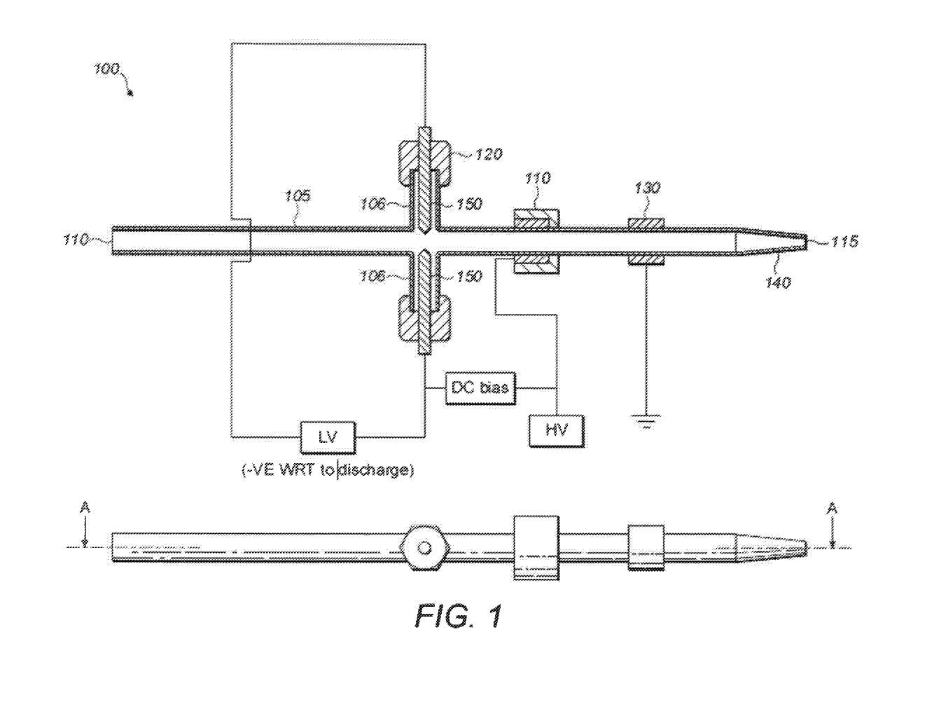

FIG. 1 shows an embodiment of a discharge device 100 in accordance with the invention.

The seeded discharge device 100 comprises: a tube 105, having an inlet 110 and an outlet 115. The tube preferably has a tapered portion 140 at the outlet 115 end.

The tube 105 may be formed of or comprise pyrex or quartz.

A first electrode 110 and a second electrode 130 may surround the tube. Preferably, the first and second electrodes 110, 130 are spaced apart along the length of the tube. Preferably, the second electrode 130 is located between the first electrode and the outlet 115.

The first electrode 110 may be shielded on its outer surface for preventing unwanted discharges. The shield may comprise or be formed of PTFE. The second electrode 130 may be earthed. Preferably, one or both of the first and second electrodes 110, 130 are made from copper foil.

Between the first electrode 110 and the inlet 110 there may be provided a pair of rod electrodes 150. The rod electrodes 150 may penetrate the tube 105.

For example, the tube 105 may include two branches 106 extending therefrom. Each rod electrode 150 may be inserted into the ends of a branch 106.

Preferably, the ends of the rod electrodes 150 are closer together then the width of the tube 105 where they penetrate the tube 105.

The rod electrodes 150 may have pointed ends.

There may be provided means for applying a voltage between the rod electrodes 150 to thereby provide a spark within the tube.

A suitable device described herein may run at 60 kHz, appearing to produce a steady afterglow and plume with short bursts of ionisation superimposed on top, presumably at 120 kHz. These ion spikes may spread as the gas flow carries them downstream, but as the field oscillates between the high voltage and earthed (quartz barrier) electrodes, the field also spreads downstream, through the afterglow to earth. This earth is either the atmosphere itself, which forms a diffuse ‘electrode’ or, when a surface gets close enough to the afterglow outlet, it becomes the earthed surface (e.g. a tooth). The field wave, on its way to ground downstream, excites the electrons carried downstream, creating continuous waves of low level ionisation all the way downstream to the target. This helps sustain charged (and presumably neutral radical) particle levels in the afterglow, and is partly why it continues to glow, judging from the emission spectra. However, whereas the discharge current is about 8 mA, the downstream current is of the order of 0.1 mA, so is small or negligible in its drain on the power supply.

To limit the average discharge power, the high frequency power source to the discharge may be gated on and off (fraction of time on is measured by the mark to space ratio) and the afterglow plasma is therefore delivered in bursts. These are separated by a few ms and diffusion is not fast enough to cause them to coalesce as they travel downstream. This is not evident to the naked eye, but it is clear from the time resolved mass spectra.

The average power level through the discharge is preferably about 3.8 watts, but the continuous power source requires about 10-12 watts.

The use of the above described electron seeding techniques allowed for a reduction of the continuous discharge current to 1-3 mA, operating at 400-500 V (rms) (560-700 V peak: i.e. 1100-1400 V p to p) whilst retaining the therapeutic dose.

The foregoing detailed description has been provided by way of explanation and illustration, and is not intended to limit the scope of the appended claims. Many variations in the presently preferred embodiments illustrated herein will be apparent to one of ordinary skill in the art, and remain within the scope of the appended claims and their equivalents.

1. A plasma-generation device for applying plasma to a human body, the device comprising:
a reservoir containing a gas;
a plasma zone in fluid connection with the reservoir;
means for generating a plasma by electrical discharge in the plasma zone,
wherein downstream of the reservoir and upstream of the plasma zone there is provided a means for generating free electrons.

2. The device of claim 1, wherein the means for generating free electrons is a radiation source.

3. The device of claim 2, wherein the radiation source comprises americanium.

4. The device of claim 1, wherein the means for generating free electrons is a UV lamp.

5. The device of claim 1, wherein the means for generating free electrons is an electrical filament.

6. The device of claim 1, wherein the means for generating free electrons comprises a pair of electrodes configured to provide a spark when a voltage is applied between them.

7. The device of claim 1, wherein the means for generating a plasma comprises a power supply and a dielectric electrode for placing in proximity to a human body, and wherein, in use, the plasma zone is formed between the dielectric electrode and a surface of a human body.

8. The device of claim 1, wherein the means for generating a plasma comprises a power supply, and first and second electrodes, and wherein, in use, the plasma zone is formed between the first and second electrodes and wherein a flow of gas from the reservoir through the plasma zone provides a flow of plasma to contact a surface of a human body.

9. The device of claim 1, wherein the means for generating a plasma comprises a power supply, and first and second electrodes sandwiching a dielectric material, and wherein, in use, the plasma zone is formed between the first or second electrode and a surface of a human body.

10. The device of claim 1, wherein the device is hand-held.

11. The device of claim 10, wherein the power supply comprises a battery integrated into the hand-held device.

12. The device of any claim 1, wherein the means for generating a plasma operates at a voltage of from 2-15 kV.

13. The device of any claim 1, wherein the device is a hair straightener, a toothbrush, foot-spa or a hair-brush.

14. The device of claim 1, wherein the gas comprises Helium, Argon, Neon, Krypton, or Hydrogen, or mixtures of two or more thereof.

15. A method for the provision of a plasma using a plasma-generation device, the device comprising a reservoir containing a gas: a plasma zone in fluid connection with the reservoir, means for generating a plasma by electrical discharge in the plasma zone; and means for generating free electrons located downstream of the reservoir and upstream of the plasma zone; the method comprising:
flowing gas from the reservoir, via the means for generating free electrons, to the plasma zone and generating a plasma by electrical discharge in the plasma zone.

16. The method of claim 15, wherein the gas is supplied through the means for generating a plasma at a flow rate of less than 5 l/min.

17. The method of claim 16, wherein the gas flow rate is less than 2.5 l/min.

18. The method of claim 17, wherein the gas flow rate is less than 1.5 l/min.

19. The method of claim 18, wherein the gas flow rate is from 0.1 to 0.5 l/min.
